(19) United States
(12) Patent Application Publication
(10) Pub. No.: US 2002/0173478 A1
(43) Pub. Date: Nov. 21, 2002

(54) Post-transcriptional gene silencing by RNAi in mammalian cells

(75) Inventor: Alan Gewirtz, Narberth, PA (US)

Correspondence Address: DILWORTH PAXSON LLP, 3200 MELLON BANK CENTER, 1735 MARKET STREET, PHILADELPHIA, PA, 19103, US

(73) Assignee: The Trustees Of The University Of Pennsylvania, Philadelphia, PA

(21) Appl. No.: 09/993,183

(22) Filed: Nov. 14, 2001

Related U.S. Application Data
(60) Provisional application No. 60/248,346, filed on Nov. 14, 2000.

(51) Int. Cl. A61K 48/00; A01K 67/00; C12N 15/85; C12Q 1/68

(52) U.S. Cl. 514/44; 800/8; 435/455; 435/6

(57) ABSTRACT

Provided is a method for disrupting cell expression at the mRNA level in mammalian cells using a post-transcriptional gene silencing method known as “RNA mediated interference” or “RNA interference” (“RNAi”). It also provides, for the first time, a demonstration of a RNAi technique that is applicable to human cells and cell lines, as well as for administration to human patients. Thus, this discovery of the value of RNAi for inhibiting mammalian cell expression offers a tool for developing new strategies for blocking gene function, and for producing RNA-based drugs to treat human disease and evaluate vaccine development targets, some of which may not be readily apparent on the basis of sequence information alone.

### RELATED APPLICATION

This application claims the benefit of U.S. Provisional Application No. 60/248,346, filed Nov. 14, 2000, which filing date is claimed herein, and the content of which is herein incorporated by reference.

### FIELD OF THE INVENTION

The invention relates to the process of disrupting cell expression at the mRNA level in mammalian cells using a post-transcriptional gene silencing method, known as RNA interference (RNAi).

### BACKGROUND OF THE INVENTION

Double-stranded ribonucleic acids (dsRNAs) are naturally rare and have been found only in certain microorganisms, such as yeasts or viruses. Until recently, dsRNA was considered only to be a molecule of essentially theoretical interest, and it was thought that its only applications were related to basic research.

However, it has since been demonstrated that dsRNAs can, transiently, be involved in phenomena of regulation of expression, as well as in the initiation of the synthesis of interferon by cells (Declerq et al., Meth. Enzymol. 78:291 (1981); Wu-Li, Biol. Chem. 265:5470 (1990)). In addition, dsRNA has been reported to have anti-proliferative properties, which makes it possible also to envisage therapeutic applications (Aubel et al., Proc. Natl. Acad. Sci., USA 88:906 (1991)). For example, synthetic dsRNA has been shown to inhibit tumor growth in mice (Levy et al. Proc. Nat. Acad. Sci. USA, 62:357-361 (1969)), is active in the treatment of leukemic mice (Zeleznick et al., Proc. Soc. Exp. Biol. Med. 130:126-128 (1969)); and inhibits chemically-induced tumorigenesis in mouse skin (Gelboin et al., Science 167:205-207 (1970)). However, when the early effects of dsRNA were first seen, the mechanism responsible for the effect was unknown, and thus, could not be well controlled. Moreover, the production of dsRNA was considered difficult.

The few known processes for preparing dsRNA can be divided into four categories:

1) Extraction of double-stranded RNA from biological material, e.g., from viruses (Boccardo et al., in Double stranded RNA viruses, 1983, Bishop Eds., Elsevier, N.Y.); Dulieu et al., J. Virol. Meth. 1989, 24, 77-84 (1989)). The presence of dsRNA in certain yeasts has been demonstrated by Fried et al., Proc. Natl. Acad. Sci. USA 75:4225 (1978) and by Al-Hakeem et al., Anal. Biochem. 163:433-439 (1987)). Among the specific sources of natural dsRNA are virus particles found in certain strains of Penicillium chrysogenum, Penicillium funiculosum, Penicillium stoloniferum, Aspergillus niger, Aspergillus foetidus, φ6 bacteriophage, and the like.

2) Hybridization of two complimentary, single-stranded RNAs (Sadher et al., Biochem. Int., 14:1015 (1987)). Each RNA chain is then synthesized by in vitro transcription of a recombinant plasmid, wherein the DNA sequence to be transcribed is positioned downstream of a promoter sequence of a DNA-dependent RNA polymerase, producing, e.g., polyriboinosinic-polyribocytidilic acid (poly I:poly C), or poly A:poly U, poly G:poly C, and the like. The single-stranded RNAs are next purified and quantified, and then hybridized to form a double strand of RNA. Methods for producing and isolating dsRNA are well recognized in the literature, see, e.g., U.S. Pat. No. 3,597,318 and U.S. Pat. No. 3,582,469.

More recently, RNA has been synthesized using transcription of a synthetic DNA template to single-stranded RNAs, which are then combined and hybridized with each other (Bhattacharyya, Nature 343:484(1990); Milligan, Nucleic Acids Res., 21:8783 (1987)). However, such techniques are relatively long and difficult to implement because prior to hybridization, they require the preparation of two recombinant or synthetic template DNAs and the purification of the two RNA strands.

3) Production of repetitive or homopolymeric dsRNA (Yano et al., French Patent Application 2 617 403). However, in no case is this technique been applicable to the synthesis of complex RNAs.

4) Synthesis of double-stranded RNA from a DNA template of given sequence. U.S. Pat. No. 5,795,715 teaches a process for the simultaneous transcription of the two complementary strands of a DNA sequence, carried out under determined conditions and in the same reaction compartment. Consequently, the two resulting transcripts hybridize immediately between themselves, giving rise to a dsRNA.

RNA interference (RNAi) is an evolutionarily conserved gene silencing mechanism, originally discovered in studies of the nematode Caenorhabditis elegans (Lee et al, Cell 75:843 (1993); Reinhart et al., Nature 403:901 (2000)). It is triggered by introducing dsRNA into cells expressing the appropriate molecular machinery, which then degrades the corresponding endogenous mRNA. The mechanism involves conversion of dsRNA into short RNAs that direct ribonucleases to homologous mRNA targets (summarized, Ruvkun, Science 2294:797 (2001)). This process is related to normal defense against viruses and the mobilization of transposons. Treatment with dsRNA has become an important method for analyzing gene functions in invertebrate organisms.

For example, Dzitoveva et al. showed for the first time, that RNAi can be induced in adult fruit flies by injecting dsRNA into the abdomen of anesthetized Drosophila, and that this method can also target genes expressed in the central nervous system (Mol. Psychiatry 6(6):665-670 (2001)). Both transgenes and endogenous genes were successfully silenced in adult Drosophila by intra-abdominal injection of their respective dsRNA. Moreover, Elbashir et al., provided evidence that the direction of dsRNA processing determines whether sense or antisense target RNA can be cleaved by a small interfering RNA (siRNA)-protein complex (Genes Dev. 15(2): 188-200 (2001)).

As shown by two recent reports, RNAi provides a rapid method to test the function of genes in the nematode Caenorhabditis elegans; and most of the genes on C. elegans chromosome I and III have now been tested for RNAi phenotypes (Barstead, Curr. Opin. Chem. Biol. 5(1):63-66 (2001); Tavernarakis, Nat. Genet. 24(2):180-183 (2000); Zamore, Nat. Struct. Biol. 8(9):746-750 (2001).). When used as a rapid approach to obtain loss-of-function information, RNAi was used to analyze a random set of ovarian transcripts and have identified 81 genes with essential roles in C. elegans embryogenesis (Piano et al., Curr. Biol. 10(24):1619-1622 (2000). RNAi has also been used to disrupt the pupal hemocyte protein of Sarcophaga (Nishikawa et al., Eur. J. Biochem. 268(20):5295-5299 (2001)).

Schoppmeier et al., Dev. Genes Evol. 211(2):76-82 (2001) developed a dsRNAi protocol for spiders while studying the function of the Distal-less gene in arthropod appendage formation.

Like RNAi in invertebrate animals, post-transcriptional gene silencing (PTGS) in plants is an RNA-degradation mechanism. In plants, this can occur at both the transcriptional and the post-transcriptional levels; however, in invertebrates only post-transcriptional RNAi has been reported to date (Bernstein et al., Nature 409(6818):295-296 (2001). Indeed, both involve double-stranded RNA (dsRNA), spread within the organism from a localized initiating area, to correlate with the accumulation of small interfering RNA (siRNA) and require putative RNA-dependent RNA polymerases, RNA helicases and proteins of unknown functions containing PAZ and Piwi domains.

However, some differences are evident between RNAi and PTGS were reported by Vaucheret et al., J. Cell Sci. 114(Pt 17):3083-3091 (2001). First, PTGS in plants requires at least two genes—SGS3 (which encodes a protein of unknown function containing a coil-coiled domain) and MET1 (which encodes a DNA-methyltransferase)—that are absent in C. elegans, and thus are not required for RNAi. Second, all of the Arabidopsis mutants that exhibit impaired PTGS are hyper-susceptible to infection by the cucumovirus CMV, indicating that PTGS participates in a mechanism for plant resistance to viruses. RNAi-mediated oncogene silencing has also been reported to confer resistance to crown gall tumorigenesis (Escobar et al., Proc. Natl. Acad. Sci. USA, 98(23):13437-13442 (2001)).

RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger, but the protein components of this activity remained unknown. Hammond et al. (Science 293(5532):1146-1150 (August 2001)) reported biochemical purification of the RNAi effector nuclease from cultured Drosophila cells, and protein microsequencing of a ribonucleoprotein complex of the active fraction showed that one constituent of this complex is a member of the Argonaute family of proteins, which are essential for gene silencing in Caenorhabditis elegans, Neurospora, and Arabidopsis. This observation suggests links between the genetic analysis of RNAi from diverse organisms and the biochemical model of RNAi that is emerging from Drosophila in vitro systems.

However, when used in vertebrate species, RNAi was found to be unpredictable, with very low efficiencies. (Fjose et al., Biotechnol. Annu. Rev. 7:31-57 (2001). For example, when tested in zebrafish embryos, RNAi was proven not to be a viable technique for studying gene function (Zhao et al., Dev. Biol. 229(1):215-223 (January 2001), yet it was effective when used in Xenopus embryos (Nakano et al., Biochem. Biophys. Res. Commun. 274(2):434-439 (2000).

Svoboda et al. reported in Development 127(19):4147-4156 (2000) that RNAi provides a suitable and robust approach to study the function of dormant maternal mRNAs in mouse oocytes. Mos (originally known as c-mos) and tissue plasminogen activator mRNAs are dormant maternal mRNAs are recruited during oocyte maturation, and translation of Mos mRNA results in the activation of MAP kinase. The dsRNA directed towards Mos or TPA mRNAs in mouse oocytes specifically reduced the targeted mRNA in both a time- and concentration-dependent manner, and inhibited the appearance of MAP kinase activity. See also, Svoboda et al. Biochem. Biophys. Res. Commun. 287(5):1099-1104 (2001).

Nevertheless, in light of the foregoing it is clear that prior to the present invention a need remained in the art for a reliable and effective method for inhibiting targeted expression in mammalian cells and cell lines. In particular, there remained a need for such a method for inhibiting the proliferation and migration of tumor cells in human patients, and for inhibiting metastatic cancer development. Moreover, such a method would have implications for functional genomics, as well as for creating functional ‘knockout’ organisms, or for tissue- and stage-specific gene targeting.

### SUMMARY OF THE INVENTION

The present invention provides a method for disrupting cell expression at the mRNA level in mammalian cells using a post-transcriptional gene silencing method known as “RNA mediated interference” or “RNA interference” (“RNAi”). It also provides, for the first time, a demonstration of the application of the RNAi technique in human cells. Thus, this discovery of the value of RNAi for inhibiting mammalian cell expression offers a tool for developing new strategies for blocking gene function, and for producing RNA-based drugs to treat human disease. It is anticipated that this technique will not only provide insights into gene function, but also help investigators to mine the genome for candidate drug intervention or vaccine development targets, some of which may not be readily apparent on the basis of sequence information alone.

The invention provides the method, wherein the cells are mammalian cells, and in one embodiment the cells are human.

Also provided is an embodiment in which the method further comprises screening dsRNAs to identify the dsRNA that disrupts target cell expression at the mRNA level. In a preferred embodiment, the target cell disrupted by the method of the invention is a tumor cell, and in another embodiment the target cell is malignant. In yet another embodiment, the method further comprises producing a ‘knock-out’ model animal in which target cell expression is disrupted at the mRNA level.

The invention also provides a method for detecting the presence of a target nucleic acid sequence in a biological sample, comprising the steps of: transcribing the target sequence into dsRNA; exposing the biological sample to the dsRNA; and detecting inhibition of gene function of the target nucleic acid sequence in the biological sample, wherein if inhibited, the target nucleic acid sequence is present in the sample. Further provided is the method, wherein at least two different target sequences are transcribed into dsRNAs and the corresponding inhibited gene expressions are detected simultaneously in the same sample.

In addition, the invention provides a method for treating a mammalian subject with an RNA-based disorder or disease by administering to the subject a dsRNA preparation for initiating disruption of target cell expression at the mRNA level, wherein the method comprises using RNAi to achieve post-transcriptional gene silencing. In this embodiment, the preferred mammalian subject is a human patient. Again, an embodied target cell in the method of the invention is a tumor cell, and the tumor cell may be malignant. Moreover, in this embodiment, as in the method above, the method further comprises initiating RNAi, wherein the dsRNA is specific for a target gene encoding the disrupted expression. The method also comprises blocking mammalian gene function of the target gene encoding the disrupted expression., as well as producing RNA-based drugs to disrupt target cell expression at the mRNA level.

Additional objects, advantages and novel features of the invention will be set forth in part in the description, examples and figures which follow, and in part will become apparent to those skilled in the art on examination of the following, or may be learned by practice of the invention.

### DESCRIPTION OF THE DRAWINGS

The foregoing summary, as well as the following detailed description of the invention, will be better understood when read in conjunction with the appended drawings. For the purpose of illustrating the invention, there are shown in the drawings, certain embodiment(s) which are presently preferred. It should be understood, however, that the invention is not limited to the precise arrangements and instrumentalities shown. FIGS. 1A-1O depict flow cytograms of c-Kit receptor expression on CHP-100 HL60 cells exposed to increasing amounts of c-Kit dsRNA. Each row has three figures indicating the dosage applied, e.g. FIGS. 1A-1C represent a set, each of which are positive controls, FIGS. 1D-1F represent a set, each of which, have a dosage of 22.5 μg/ml, etc. FIGS. 1A, 1D, 1G, 1J and 1M are histograms of cells being analyzed. X axis is “forward light scatter” [size index]; Y axis is “side scatter” [internal content]. The circle indicates the cells chosen for further analysis, in the middle column of figures (FIGS. 1B, 1E, 1H, 1K and 1N). FIGS. 1B, 1E, 1H, 1K and 1N show cells labeled with control antibody (fluoroscene labeled). X axis is fluoresence intensity in log scale, Y axis is number of events, i.e. cell counted. The left most figure is a superimposition of the control histogram (displayed as “open” or white) and histogram of cells labeled with Anti-c-Kit Receptor antibody. In FIGS. 1C, 1F, 1I, 1L and 1O fluorescence intensity of cells labeled with isotype control antibody is shown by “open” histogram. C-Kit positive (+) cells are shown in shaded histogram. Note leftward shift of histogram in response to increasing concentration of dsKit RNA.

FIGS. 2A-2L depict flow cytograms of c-Kit receptor expression on HL60 cells exposed to increasing amounts of c-Kit dsRNA for 4 days of incubation. As in FIG. 1, each row has three figures indicating the dosage applied, e.g. FIGS. 1A-1C represent a set, each of which are positive controls, FIGS. 1D-1F represent a set, each of which, have a dosage of 70 μg, etc. FIGS. 1A, 1D, 1G, and 1J are histograms of cells being analyzed. X axis is “forward light scatter” [size index]; Y axis is “side scatter” [internal content]. The circle indicates the cells chosen for further analysis, in the middle column of figures (FIGS. 1B, 1E, 1H, and 1K). FIGS. 1B, 1E, 1H, and 1K show cells labeled with control antibody (fluoroscene labeled). X axis is fluoresence intensity in log scale, Y axis is number of events, i.e. cell counted. The left most figure is a superimposition of the control histogram (displayed as “open” or white) and histogram of cells labeled with Anti-c-Kit Receptor antibody. In FIGS. 1C, 1F, 1I, and 1L fluorescence intensity of cells labeled with isotype control antibody is shown by “open” histogram. C-Kit positive (+) cells are shown in shaded histogram. Note leftward shift of histogram in response to increasing concentration of dsKit RNA.

FIG. 3 photographically depicts an agarose gel showing the effect of dsRNA on c-Kit receptor signaling in HL-60 cells.

### DESCRIPTION OF PREFERRED EMBODIMENTS OF THE INVENTION

The invention provides, for the first time, evidence of post-transcriptional gene silencing by dsRNA interference (RNAi) for inhibiting targeted expression in mammalian cells at the mRNA level, more importantly in human cells or cell lines. It further provides a reliable and effective method for inhibiting the proliferation and migration of tumor cells in human patients, and for inhibiting metastatic cancer development. Thus, the method of the invention is of particular significance in vivo in a human patient. Moreover, such a method has implications for functional genomics, as well as for creating functional ‘knockout’ organisms, or for tissue- and stage-specific gene targeting.

By “target gene” is meant a targeted nucleic acid sequence, the expression of which is being silenced in the present invention by RNAi. The “target cell” therefore, is the cell from which the target gene is expressed, and in which the gene expression is disrupted by RNAi, wherein exposure to dsRNA homologous to the target gene initiates the disruption. The disruption is detected and measurable in terms of “inhibition” or reduction of the expression of the target gene, which is reflected in terms of a reduction or decrease of activity of the expression product, as compared with the activity, absent treatment with the homologous dsRNA, from the targeted gene.

Subsequent to disclosure of the present invention, Yang et al., Mol. Cell Biol. 21(22):7807-7816 (2001) reported the lack of success by others who have attempted to use RNAi in mammalian systems. Yang et al. investigated the feasibility of the RNAi strategy in several mammalian cells by using the enhanced green fluorescent protein gene as a target, following the method of the present invention, either by in situ production of dsRNA from transient transfection of a plasmid harboring a 547-bp inverted repeat, or by direct transfection of dsRNA made by in vitro transcription. Several mammalian cells, including differentiated embryonic stem (ES) cells, did not exhibit specific RNAi in transient transfection. This long dsRNA, however, was capable of inducing a sequence-specific RNAi for the episomal and chromosomal target gene in undifferentiated mouse ES cells, and cognate gene expression was decreased up to 70%. However, RNAi activity was not permanent. It was pronounced in early time points, but its activity diminished significantly by the 5^{th} day after transfection.

In both plants and animals, RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger, although the protein components of this activity are unknown. However, the 22-nucleotide RNA sequences are homologous to the target gene that is being suppressed. Thus, the 22-nucleotide sequences appear to serve as guide sequences to instruct a multicomponent nuclease, RISC, to destroy the specific mRNAs.

Carthew also reported (Curr. Opin. Cell Biol. 13(2):244-248 (April 2001) (following disclosure of the present invention), that eukaryotes silence gene expression in the presence of dsRNA homologous to the silenced gene. Biochemical reactions that recapitulate this phenomenon generate RNA fragments of 21 to 23 nucleotides from the double-stranded RNA. These stably associate with an RNA endonuclease, and probably serve as a discriminator to select mRNAs. Once selected, mRNAs are cleaved at sites 21 to 23 nucleotides apart.

The dsRNA used to initiate RNAi, may be isolated from native source or produced by known means, e.g., transcribed from DNA. For example, the binding of an RNA polymerase to a promoter (meaning any double-stranded sequence of DNA comprising a binding site recognized by a DNA-dependent RNA polymerase) permits initiation of transcription. Many known promoter sequences can be used to produce the dsRNA, for example, but limited to, the sequences recognized by the RNA polymerases of phages T7, T3 or SP6. This does not, however, represent a limitation, because it will appear clearly to a person skilled in the art that any promoter sequence identified as such, and for which the corresponding RNA polymerase is available, can be used.

Alternatively, the two strands of DNA used to form the dsRNA may belong to the same or two different duplexes in which they each form with a DNA strand of at least partially complementary sequence. When the dsRNA is thus-produced, the DNA sequence to be transcribed is flanked by two promoters, one controlling the transcription of one of the strands, and the other that of the complementary strand. These two promoters may be identical or different. In fact, in accordance with U.S. Pat. No. 5,795,715, a DNA duplex provided at each end with a promoter sequence can directly generate RNAs of defined length, and which can join in pairs to form a dsRNA.

The dsRNA, whether of synthetic or natural origin, is subject to rapid degradation by nucleases present in the sera of various animal species, particularly primates. Consequently procedures involving dsRNA generally utilize baked glassware throughout, and all buffers are filtered, e.g., through a Nalgene 45 micron filter, for sterility. Pyrogen-free, double distilled water must be used for all solutions to minimize any possibility of endotoxin contamination.

The concentration of the dsRNA solution may be determined from its UV spectrum. For example, the molar concentration of natural or synthetic dsRNA is determined from the optical density (OD) at 260 nm using an extinction coefficient, obtainable from the literature or determined using standard procedures: 44.7 times OD₂₆₀=micrograms dsRNA/ml.

If appropriate, the dsRNA solution can be diluted with pyrogen-free buffer for ease in handling.

The resulting dsRNA can optionally be linked to a support; or to a ligand, such as biotin, which can be attached to a support coated with avidin. This permits direct quantification, when utilized as an analytical tool.

In one embodiment, the dsRNA compositions of the present invention are prepared as pharmaceutical composition for the treatment of subjects, particularly for the treatment of human patients. More particularly the pharmaceutical compositions are administered to inhibit the proliferation and migration of tumor cells in human patients, particularly malignant tumors, and for inhibiting metastatic cancer development. In an alternative embodiment, the compositions are used to create functional ‘knockout’ model organisms, those in which a target gene is defective, or in this case, expression is inhibited.

The dsRNA pharmaceutical compositions of the present invention preferably contain a pharmaceutically acceptable carrier or excipient suitable for rendering the compound or mixture administrable orally as a tablet, capsule or pill, or parenterally, intravenously, intradermally, intramuscularly or subcutaneously, or transdermally. The active ingredients may be admixed or compounded with any conventional, pharmaceutically acceptable carrier or excipient.

As used herein, the term “pharmaceutically acceptable carrier” includes any and all solvents, dispersion media, coatings, antibacterial and antifungal agents, isotonic agents, absorption delaying agents, and the like. The use of such media and agents for pharmaceutically active substances is well known in the art. Except insofar as any conventional media or agent is incompatible with the compositions of this invention, its use in the therapeutic formulation is contemplated. Supplementary active ingredients can also be incorporated into the pharmaceutical formulations.

It will be understood by those skilled in the art that any mode of administration, vehicle or carrier conventionally employed and which is inert with respect to the active agent may be utilized for preparing and administering the pharmaceutical compositions of the present invention. Illustrative of such methods, vehicles and carriers are those described, for example, in Remington's Pharmaceutical Sciences, 4th ed. (1970), the disclosure of which is incorporated herein by reference. Those skilled in the art, having been exposed to the principles of the invention, will experience no difficulty in determining suitable and appropriate vehicles, excipients and carriers or in compounding the active ingredients therewith to form the pharmaceutical compositions of the invention.

The therapeutically effective amount of active agent to be included in the pharmaceutical composition of the invention depends, in each case, upon several factors, e.g., the type, size and condition of the patient to be treated, the intended mode of administration, the capacity of the patient to incorporate the intended dosage form, etc. Generally, an amount of active agent is included in each dosage form to provide from about 0.1 to about 250 mg/kg, and preferably from about 0.1 to about 100 mg/kg.

While it is possible for the agents to be administered as the raw substances, it is preferable, in view of their potency, to present them as a pharmaceutical formulation. The formulations of the present invention for human use comprise the agent, together with one or more acceptable carriers therefor and optionally other therapeutic ingredients. The carrier(s) must be “acceptable” in the sense of being compatible with the other ingredients of the formulation and not deleterious to the recipient thereof. Desirably, the formulations should not include oxidizing agents and other substances with which the agents are known to be incompatible. The formulations may conveniently be presented in unit dosage form and may be prepared by any of the methods well known in the art of pharmacy. All methods include the step of bringing into association the agent with the carrier, which constitutes one or more accessory ingredients. In general, the formulations are prepared by uniformly and intimately bringing into association the agent with the carrier(s) and then, if necessary, dividing the product into unit dosages thereof.

Formulations suitable for parenteral administration conveniently comprise sterile aqueous preparations of the agents, which are preferably isotonic with the blood of the recipient. Suitable such carrier solutions include phosphate buffered saline, saline, water, lactated ringers or dextrose (5% in water). Such formulations may be conveniently prepared by admixing the agent with water to produce a solution or suspension, which is filled into a sterile container and sealed against bacterial contamination. Preferably, sterile materials are used under aseptic manufacturing conditions to avoid the need for terminal sterilization.

Such formulations may optionally contain one or more additional ingredients among which may be mentioned preservatives, such as methyl hydroxybenzoate, chlorocresol, metacresol, phenol and benzalkonium chloride. Such materials are of special value when the formulations are presented in multidose containers.

Buffers may also be included to provide a suitable pH value for the formulation. Suitable such materials include sodium phosphate and acetate. Sodium chloride or glycerin may be used to render a formulation isotonic with the blood. If desired, the formulation may be filled into the containers under an inert atmosphere such as nitrogen or may contain an anti-oxidant, and are conveniently presented in unit dose or multi-dose form, for example, in a sealed ampoule.

The invention is further described by example. The examples, however, are provided for purposes of illustration to those skilled in the art, and are not intended to be limiting. Moreover, the examples are not to be construed as limiting the scope of the appended claims. Thus, the invention should in no way be construed as being limited to the following examples, but rather, should be construed to encompass any and all variations that become evident as a result of the teaching provided herein.

### EXAMPLES

To demonstrate the effectiveness of RNAi as an efficient, and highly reproducible, strategy for disrupting gene expression at the mRNA level in mammalian cells, the effect of dsRNA was evaluated in a receptive cell. Using the c-Kit gene as a target, the effect of dsRNA on the expression of the c-kit receptor (KitR) was evaluated in malignant human neuroepithelial and hematopoietic cells.

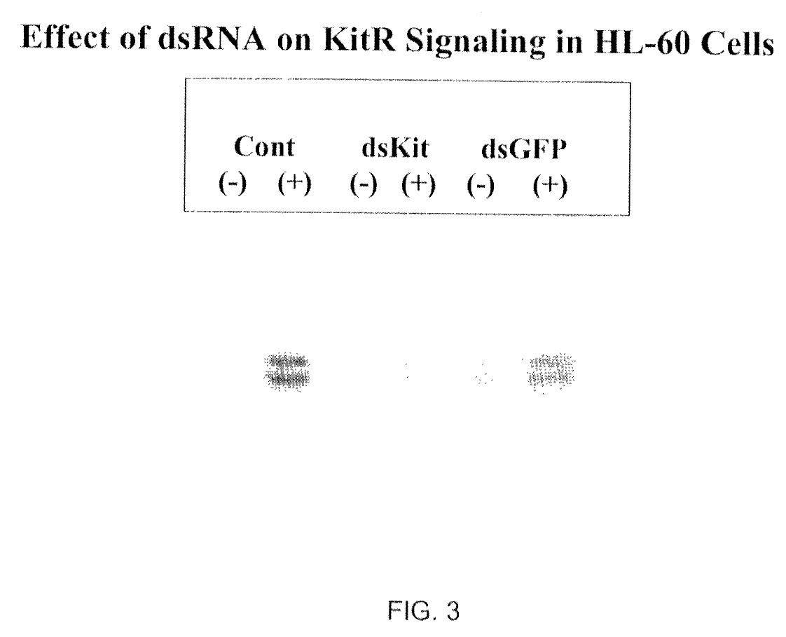

To begin, 828 bp (nucleotide base pairs) of the 5′ end of c-Kit genomic cDNA was subcloned into expression vector pcDNA3 (containing T7 and SP6 promoters) by digesting with BamH1. As a control, 724 bp of Green Fluorescent Protein (GFP) cDNA was subcloned into pcDNA3 by digesting with EcoRI and HindIII. Subcloned vectors were amplified in a chemically competent strain of E. coli DH5α cells. In vitro transcription reactions were carried out using known methods to linearize the plasmids using EcoRV and HindIII to synthesize the sense and corresponding antisense RNA strands, respectively. Digested plasmids were treated with Proteinase K to inactive any RNases, and purified as a template for transcription by QIAquick Purification Kit (Qiagen). RNA polymerases were from Promega. The products were pooled and annealed for 10 minutes at 90° C., 10 minutes at 4° C., and 2 hours at 40° C. in a hybridization mixture containing NaCl 250 mM, Tris HCl 40 mM at pH 7.5 and EDTA 5 mM in RNase free water. The dsRNA was eluted using diethyl pyrocarbonate treated H₂O, and the integrity of the dsRNA was confirmed by running a 1% agarose gel in TBE 1X (90 mM Tris-borate/2 mM EDTA pH 8.0). It was then purified by column chromatography. See, for example, FIG. 3 depicting an agarose gel showing the affect of dsRNA on c-Kit receptor signaling in HL-60 cells.

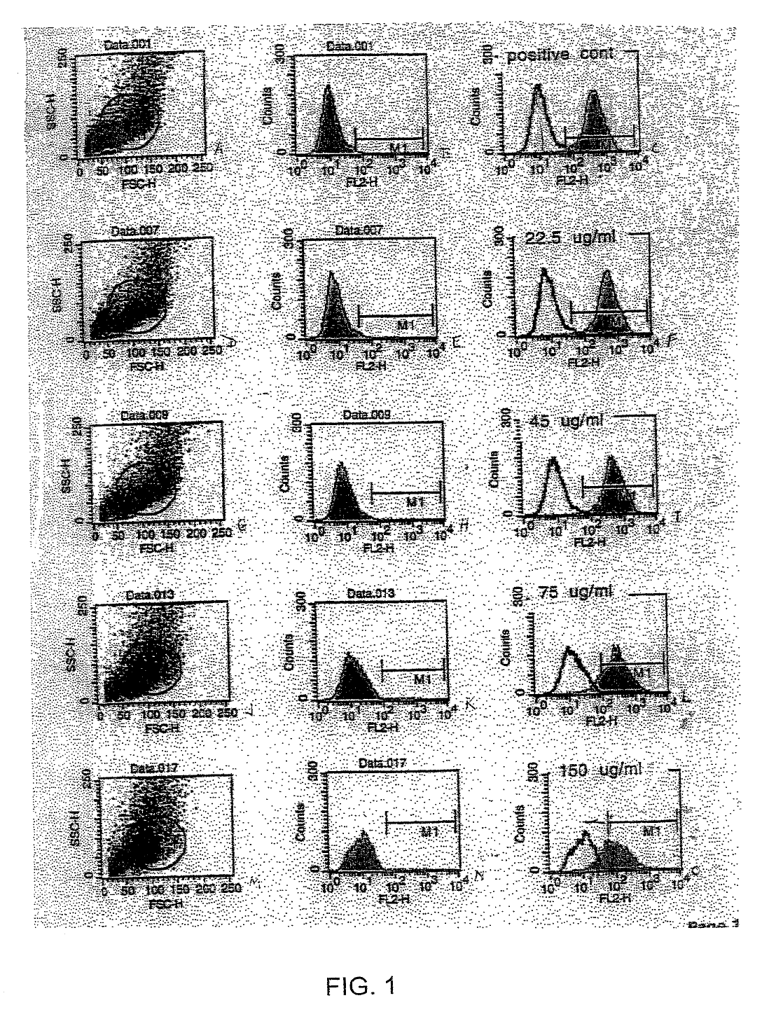

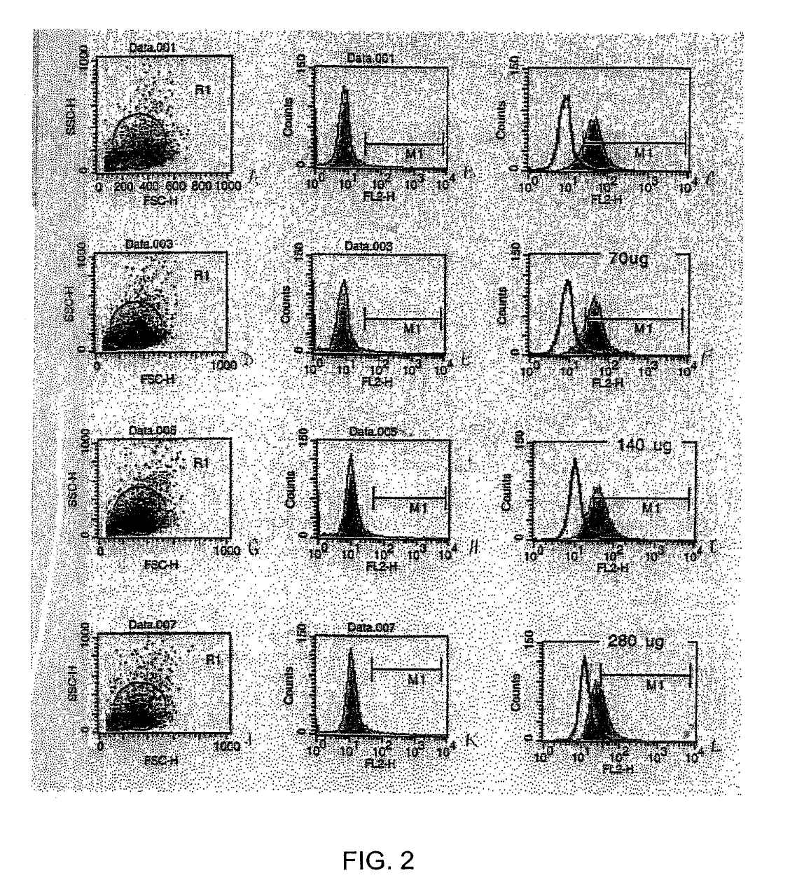

CHP 100 neuroepithelioma (human melanoma) cells and HL-60 (human leukemia) cells are both known to express KitR, and therefore, were employed as indicator cells. Cell lines were maintained in RPMI 1640 media (GibcoBRL, Gaithersburg, Md.) containing 10% BCS. Varying amounts (150-350 μg/ml) of Kit dsRNA (KdsRNA) were added to the culture media, or as a control, GFP dsRNA (GdsRNA) was added. Cells were incubated under the same conditions, at 37° C., in 5% CO₂ for 1-4 days. At the end of the incubation period, the cells were washed with PBS (phosphate buffered saline) and detached from the culture vessels using versene. FACS analyses were performed immediately and the results are shown in FIGS. 1 and 2, as described in the description of drawings.

All FACS acquisitions were performed using CELLquest software (Becton Dickenson) on a FACScan flow cytometer. The c-Kit cells were stained to determine KitR expression using a 1:1000 dilution of a c-Kit monoclonal antibody (Dr. Virginia Brody, Univ. of Washington, Seattle, Wash.). Isotypic control antibody was obtained from DAKO. All antibodies were used at saturating concentrations and cells were incubated for 30 minutes on ice, followed by two washes with PBS.

No effect on KitR expression was observed until day 3, but then inhibition was seen, although only in the cells exposed to the KdsRNA. For example, after incubation with 150 μg/ml of c-KdsRNA, the percentage of (+) CHP cells decreased from its initial expression of 96±2% to 80±3%. The mean geometric fluorescence intensity on expressing cells decreased 2.25+0.25 fold (p<0.01).

After incubation with a dose of 250 μg/ml of KdsRNA, KitR was decreased to 67±2%, and the mean geometric fluorescence intensity decreased by 2.75±0.50 fold (p<0.01). Dose levels of KdsRNA <150 μg/ml had no effect on c-Kit expression; and dose levels >350 μg/ml of dsRNA were toxic to the mammalian cells. The results are summarized in Table 1.

TABLE 1
Effect of KdsRNA and GdsRNA on c-Kit expression in CHP 100 cells.

| | % c-Kit expression | | | |
|---|---|---|---|---|
| | on CHP 100 cells | | Changes in geometric mean | P |
| | Initial | Day 3 | of fluorescence intensity | value |
| KdsRNA | | | | |
| Control | 97 ± 1 | 96 ± 2 | No change | |
| 150 μg/ml | 97 ± 2 | 80 ± 3 | 2.25 + 0.25 fold decrease | p<0.01 |
| 250 μg/ml | 98 ± 2 | 67 ± 2 | 2.75 + 0.50 fold decrease | p<0.01 |
| GdsRNA | | | | |
| | 96 ± 2 | 97 ± 1 | No change | NS |
| | 99 ± 3 | 98 ± 2 | No change | NS |
| | 99 ± 3 | 98 ± 2 | No change | NS |

* NS: not significant
** Results represent the average of three different experiments.

HL 60 cells behaved differently. KdsRNA doses <280 μg/ml were ineffective, but at that dose the % of (+) cells decreased dramatically from 84±2% to 36±2%. However, fluorescence intensity decreased only 1.38+0.5 fold. As was true for CHP cells, KitR expression was unaffected by comparable doses of GdsRNA.

The Kit receptor's mRNA was the target of the dsRNA. Receptor expression at the protein level (fluorescence labeling) would be expected to go down (approach the control) if the RNA was successfully targeted. This is shown in dose response manner for both.

To further document KitR disruption in HL-60 cells, the ability of Lyn kinase to be autophosphorylated was investigated. This is a known downstream effect of KitR engagement. Cells were treated with 250 μg/ml of KdsRNA for 72 hours, and then stimulated with stem cell factor (SCF) (150 ng/ml) for 10 minutes. Notably, both c-kit and SCF play important roles in follicular development in the mammalian (rodent) ovary. Cells were washed and then immediately assayed for Lyn kinase activity. When compared with controls, Lyn autophosphorylation was significantly diminished in the KdsRNA treated cells. Thus, mammalian cells are variably, but reproducibly, susceptible to RNAi, and the data support the development of therapeutically motivated PTGS in patients with malignant disease.

Each and every patent, patent application and publication that is cited in the foregoing specification is herein incorporated by reference in its entirety.

While the foregoing specification has been described with regard to certain preferred embodiments, and many details have been set forth for the purpose of illustration, it will be apparent to those skilled in the art that the invention may be subject to various modifications and additional embodiments, and that certain of the details described herein can be varied considerably without departing from the spirit and scope of the invention. Such modifications, equivalent variations and additional embodiments are also intended to fall within the scope of the appended claims.

I claim:

1. A method for disrupting target cell expression at the mRNA level in a mammalian cell, wherein the method comprises using RNA interference (RNAi) to achieve post-transcriptional gene silencing.

2. The method of claim 1, wherein the mammalian cell is from a cell line.

3. The method of claim 1, wherein the mammalian cell is a human cell.

4. The method of claim 1, wherein the method further comprises initiating RNAi in the cell by exposing the cell to a gene-specific double stranded RNA (dsRNA), wherein the dsRNA is specific for the target gene encoding the disrupted expression.

5. The method of claim 4, wherein the method further comprises blocking mammalian gene function of the target gene encoding the disrupted expression.

6. The method of claim 5, wherein the method further comprises screening dsRNAs to identify the dsRNA that disrupts target cell expression at the mRNA level.

7. The method of claim 1, wherein the target cell is a tumor cell.

8. The method of claim 7, wherein the target cell is malignant.

9. The method of claim 6, wherein the method further comprises producing RNA-based drugs to disrupt target cell expression at the mRNA level.

10. The method of claim 5, wherein the method further comprises producing a ‘knock-out’ model animal in which target cell expression is disrupted at the mRNA level.

11. The method of claim 6, wherein the RNA-based drugs that disrupt target cell expression at the mRNA level, treat human disease.

12. A method for detecting the presence of a target nucleic acid sequence in a biological sample, comprising the steps of:
transcribing the target sequence into dsRNA,
exposing the biological sample to the dsRNA, and
detecting inhibition of gene function of the target nucleic acid sequence in the biological sample, wherein if inhibited, the target nucleic acid sequence is present in the sample.

13. The method of claim 12, wherein at least two different target sequences are transcribed into dsRNAs and the corresponding inhibited gene expressions are detected simultaneously in the same sample.

14. A method for treating a mammalian subject with an RNA-based disorder or disease by administering to the subject a dsRNA preparation for initiating disruption of target cell expression at the mRNA level, wherein the method comprises using RNAi to achieve post-transcriptional gene silencing.

15. The method of claim 14, wherein the mammalian subject is a human patient.

16. The method of claim 14, wherein the method further comprises initiating RNAi, wherein the dsRNA is specific for a target gene encoding the disrupted expression.

17. The method of claim 16, wherein the method further comprises blocking mammalian gene function of the target gene encoding the disrupted expression.

18. The method of claim 17, wherein the target cell is a tumor cell.

19. The method of claim 18, wherein the target cell is malignant.

20. The method of claim 17, wherein the method further comprises producing RNA-based drugs to disrupt target cell expression at the mRNA level.
